(19) United States
(12) Patent Application Publication
(10) Pub. No.: US 2004/0171068 A1
(43) Pub. Date: Sep. 2, 2004

(54) Method for producing stable, regeneratable antibody arrays

(76) Inventors: Juergen Wehland, Braunschweig (DE); Ronald Frank, Braunschweig (DE)

Correspondence Address: James P Zeller, Marshall Gerstein & Borun, 6300 Sears Tower, 233 South Wacker Drive, Chicago, IL, 60606-6357, US

(21) Appl. No.: 10/475,147

(22) PCT Filed: Apr. 18, 2002
(86) PCT No.: PCT/EP02/04311

(30) Foreign Application Priority Data
Apr. 19, 2001 (DE) 101 19 308.4
Dec. 18, 2001 (DE) 101 62 365.8

(51) Int. Cl. G01N 33/53; C12M 1/34; B05D 3/00

(52) U.S. Cl. 435/7.1; 435/287.2; 427/2.11

(57) ABSTRACT

The invention relates to a method of producing stable, regeneratable antibody arrays using immobilised antibody-binding proteins capable of specifically identifying the Fc portion of antibodies.

The invention relates to a method of producing stable, regeneratable antibody arrays using immobilised antibody-binding proteins capable of specifically identifying the Fc portion of antibodies.

Collections of large numbers of different test compounds that are deposited/immobilised in an ordered manner on a flat surface are known in scientific terminology as arrays; see, for example, EP 0 373 203 and EP 0 619 321. Such arrays allow rapid simultaneous testing of all compounds by interaction analysis, more specifically with an analyte or a mixture of analytes in biological samples. The advantage of an array over the simultaneous testing of immobilised test compounds on mobile elements, such as, for example, beads, is that in an array the nature (chemical structure and/or identity) of the immobilised test molecules is known exactly by the location in the array surface and accordingly a local test signal can immediately be assigned to a type of molecule. Particularly in miniaturised form, arrays with biological test molecules are also referred to as biochips.

Well-established examples of such arrays are:

Nucleic acid arrays of DNA fragments, cDNAs, RNAs, PCR products, plasmids, bacteriophages, synthetic oligonucleotides or synthetic PNA oligomers, which are selected by means of hybridisation (formation of a double strand molecule) with complementary nucleic acid analytes, and compound arrays of synthetic peptides, their analogues, such as peptoids, oligocarbamates etc. or generally organic chemical compounds, which are selected by means of binding to affinitive protein analytes or to other analytes or by means of enzymatic reaction.

In contrast, protein arrays of antibodies and of proteins and phage fusion proteins expressed in cells (phage display) are still at the development stage (see below). Such arrays and the methods and apparatus developed therefor are used in basic biological research, but especially also in medical diagnostics and the development of pharmaceutical active ingredients. Other branches of scientific research, such as, for example, catalyst development and material sciences, are also beginning successfully to adopt such concepts. A prerequisite for the advantageous routine use of such arrays is their cost-effective, rapid and fully automated production with a high density and diversity of test structures (information content).

Such arrays are currently produced in accordance with two different principles by deposition of the test molecules onto pre-prepared material surfaces:

a) by a single distribution of the solution of pre-prepared test compounds on the surface

b) by repeated serial distribution of solutions of building blocks for the chemical synthesis of the test compounds in situ on the surface.

An up-to-date overview is given by S. Wöffl in: transcript Laborwelt 2000, 3, 13-20).

Chip configurations known hitherto use either a rectangular x/y arrangement, which is produced with suitably manufactured photolithographic or printing masks, or a circular rφ arrangement which is produced by a rotational movement of the chip surface (rφ arrays) and a fast-pulse dispensing device. With such configurations it is possible to achieve densities of up to 1 million test compounds per cm² or an individual surface area of a few square micrometres.

DNA arrays have proved the effectiveness of this method in many fields of biomedical research (for an overview article see Khan et al. in Biochim. Biophys. Acta 1999, 1423: 1117-1128; DeRisi et al. in Nat. Genet. 1996, 14: 457-460; Debouck and Goodfellow in Nat. Genet. 1999, 21, 48-50). There is therefore an understandable need for technologies that allow highly parallelised detection and quantification of specific proteins on the basis of a rapid and inexpensive test in a small volume format. A prerequisite for this is the establishment of highly specific, stable and regeneratable protein arrays or protein chips, for which conventional, monoclonal antibodies are extremely suitable. Hybridoma technology has long been established and standardised and yields antibodies having the desired specificity, affinity and stability.

The invention accordingly relates to a method of producing stable, regeneratable antibody arrays, in which

(a) antibody-binding proteins capable of specifically identifying the Fc portion of antibodies are covalently immobilised on the surface of a planar support,

(b) a plurality of specific monoclonal antibodies, arranged so as to form a pattern, are bound by their Fc portion to the antibody-binding proteins, and

(c) the immobilised antibody-binding protein/antibody complexes are covalently crosslinked.

The invention relates also to an antibody array obtainable in accordance with the method of the invention, to a medical or diagnostic apparatus having an antibody array according to the invention, and to a kit comprising an antibody array according to the invention as well as detection reagents for the qualitative or quantitative determination of bound antigens that have been bound to an antibody array according to the invention.

The invention further provides the use of an antibody array according to the invention or of a medical or diagnostic apparatus according to the invention for the qualitative or quantitative determination of antigens.

The subsidiary claims relate to advantageous and/or preferred embodiments of the invention.

In accordance with an embodiment of the invention, the planar support has a surface of glass, metal, metal oxides, semi-metal oxides or plastics

In accordance with a further embodiment of the invention, the antibody-binding protein is selected from Fc-specific secondary antibodies, protein A and protein G.

In accordance with an embodiment of the use indicated according to the invention, the antigen to be determined is a protein.

The invention is described in greater detail, without limitation, below.

The new production method is distinguished by the following features:

a) The specific antibodies are immobilised in a “targetted” manner, that is to say by way of their Fc portion, in order that the coupling does not affect antigen identification. For this purpose, a grid of proteins that specifically identify the Fc portion of the specific antibodies is covalently bound to the chip surface in question (for example, derivatised Fc-specific secondary antibodies or protein A or protein G molecules).

b) The necessary stabilisation of the immobilised protein/antibody complexes or antibody/antibody complexes is achieved by chemical covalent crosslinking, for which customary reagents are used in accordance with the requirements. In addition to the stabilisation of the protein-protein interactions, intramolecular stabilisation of the specific antibodies also takes place, that is to say a chemical crosslinking of their sub-units. Antibody arrays having extremely high stability are obtained which on the one hand prevent dissociation of the specific antibodies, e.g. during storage, but on the other hand also enable the antibody arrays to be treated under stringent conditions, such as high salt concentrations or a low or high pH value, in order to prevent non-specific or low-affinity interactions with the antibody matrix. As a result, pre-treatment of the protein mixtures being analysed can also be correspondingly stringent.

c) The use of covalently crosslinked antibodies requires that the antigen-binding site of the antibody in question is not deactivated or modified by the crosslinking reagent. As a consequence, therefore, there are formed or selected monoclonal antibodies the antigen-binding properties of which are not affected by the crosslinking reagent being used. For crosslinking, reference is made, for example, to Wehland & Weber in J. Cell Biol., 104 (1987) 1059.

The entire process yields stable and regeneratable antibody arrays.

1. Method of producing stable, regeneratable antibody arrays, in which
(a) antibody-binding proteins capable of specifically identifying the Fc portion of antibodies are covalently immobilised on the surface of a planar support,
(b) a plurality of specific monoclonal antibodies, arranged so as to form a pattern, are bound by their Fc portion to the antibody-binding proteins, and
(c) the immobilised antibody-binding protein/antibody complexes are covalently crosslinked.

2. Method according to claim 1, wherein the planar support has a surface of glass, metal, metal oxides, semi-metal oxides or plastics.

3. Method according to claim 1 or 2, wherein the antibody-binding protein is selected from Fc-specific secondary antibodies, protein A and protein G.

4. Antibody array, obtainable by a method according to any one of claims 1 to 3.

5. Medical or diagnostic apparatus having an antibody array according to claim 4.

6. Use of an antibody array according to claim 4 or of a medical or diagnostic apparatus according to claim 5 for the qualitative or quantitative determination of antigens.

7. Use according to claim 6, wherein the antigen to be determined is a protein.

8. Kit, comprising an antibody array according to claim 4 and detection reagents for the qualitative or quantitative determination of bound antigens.
